(19) United States
(12) Patent Application Publication
(10) Pub. No.: US 2019/0314210 A1
(43) Pub. Date: Oct. 17, 2019

(54) Protective Swatch

(71) Applicants: Giovanny Sosa Guerra, Las Vegas, NV (US); Yanelys Martinez Rifat, Las Vegas, NV (US)

(72) Inventors: Giovanny Sosa Guerra, Las Vegas, NV (US); Yanelys Martinez Rifat, Las Vegas, NV (US)

(21) Appl. No.: 16/375,796

(22) Filed: Apr. 4, 2019

Related U.S. Application Data
(60) Provisional application No. 62/762,031, filed on Apr. 17, 2018.

(51) Int. Cl. A61F 13/15; A61F 13/56

(52) U.S. Cl. CPC A61F 13/15203; A61F 2013/15065; A61F 2013/1556; A61F 13/5611

(57) ABSTRACT

A protective swatch implement including a waterproof swatch base pad that is configured to be operable for preventing a private area of a wearer from exposure or contact from infections or disease caused by germs, bacteria, and parasites that inhabit swimming pool water or natural bodies of water; a triangular-shaped notch segment is configured to be operable to allow movement without pulling when sitting and walking; an adhesive implement on a perimeter of the waterproof swatch base pad is configured to secure the waterproof swatch base pad to the private area of the wearer; an absorbent pad implement on a proximate center of the waterproof swatch base pad is configured to absorb a discharge or menstrual fluid from exiting the waterproof swatch base pad; and a display area operable for bearing at least one of, a pattern, a design, and a logo.

### CROSS-REFERENCE TO RELATED APPLICATIONS

The present Utility patent application claims priority benefit of the U.S. provisional application for patent Ser. No. 62/762,031 entitled “Hello Protection”, filed on Apr. 17, 2018, under 35 U.S.C. 119(e). The contents of this related provisional application are incorporated herein by reference for all purposes to the extent that such subject matter is not inconsistent herewith or limiting hereof.

### RELATED CO-PENDING U.S. PATENT APPLICATIONS

Not applicable.

### INCORPORATION BY REFERENCE OF SEQUENCE LISTING PROVIDED AS ATEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to water activity safety devices. More particularly, certain embodiments of the invention relate to feminine protective pad devices.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon. Water activity safety is well-known and much-practiced. Beware of the riptide at the beach, and for swimming pools, never dive unless you're sure of the depth. In all water environments, never swim alone. These and other measures are regularly employed, and help save millions from accidents and injuries. There are other risks that remain associated with natural bodies of water and swimming pools, and even though they're very well-known and for quite some time. And, it's women who are subject to these risks, where those same women might create risks for others in the water.

The following is an example of a specific aspect in related technologies that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that a trip to the beach and its natural waters may turn into a trip to the hospital due to exposure to cryptosporidium, tapeworm, hookworm, shigella, norovirus, and many other undesirables. Keeping the head above the water may not help. Infectious agents invade other personal parts of the body, including the vagina. Opting for the swimming pool won't help, either, because Staphylococcus aureus, Escherichia coli, and other infectious bacteria may be present in those waters, as well. In addition, other swimmers may deposit unwanted germs in pools, too, through their saliva, mucus, and urine.

While swimming pools may commonly carry chemicals that are supposed to prevent germs and bacteria from growing in their waters, those chemicals are known to have negative effects on the vagina. Chlorine may diminish the pH balance needed, for example, and cause an uncomfortable dryness. It may also kill the natural bacteria of Lactobacillus, which is needed for proper vaginal health. Sanitary napkins or pads have been developed for external wear about a vulvar region of a user while tampons have been developed for residence within the vaginal cavity and interruption of menstrual flow therefrom. However, these typical sanitary pads are not suitable for wearing while swimming because the pads absorb a lot of water, become bulky, leak, and are unsightly and uncomfortable when worn in a swimsuit. Tampon usage in the United States has steadily declined over the last 15 years, a recent study found. Women complain they are uncomfortable and worry about health risks associated with their use. They don't trust bulky sanitary pads, either, which women fear could be visible to others. As a result, use of menstrual cups is rapidly accelerating in the U.S. and other countries. They're not visible, and don't require regular changing, either. Menstrual cups aren't comfortable to many, however, and are only used on days of regular and heavy menstrual flow. For the days before and after, though? When risks of light discharge and spotting are present? These same cup-using women have to resort to pads.

Accordingly, there is a need for sanitary protection specifically for use by woman of all ages while swimming or participating in water sports and activities. The need has therefore been recognized for a feminine protective pad which may obviate the foregoing and other limitations and disadvantages of typical sanitary pads. Furthermore, there is a need for a protective device that may completely block undesirable germs, bacteria, and parasites from any contact with the most personal body part.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is an illustration of an exemplary protective swatch, in accordance with an embodiment of the present invention;

FIG. 2a and FIG. 2b is an illustration of an exemplary protective swatch with covering, in accordance with an embodiment of the present invention;

FIG. 3 is an illustration of an exemplary protective swatch base ready for use, in accordance with an embodiment of the present invention;

FIG. 4 is an illustration of an exemplary protective swatch base in use, in accordance with an embodiment of the present invention; and

FIG. 5 is an illustration of an exemplary protective swatch base in use, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [”substantially“] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognize in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of Claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, are generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising” And “contain” and variations of them—Such terms are open-ended and mean “including but not limited to”. When employed in the appended claims, this term does not foreclose additional structure or steps. Consider a claim that recites: “A memory controller comprising a system cache . . . .” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . ”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components is described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

Some embodiments of the present invention and variations thereof, relate to feminine protective pads. In one embodiment of the present invention, the protective swatch is operable for placement over a woman's crotch and that fully covers the vulva, protecting it and the vagina from direct contact with water during water activities, and protecting from direct contact with undesirable agents in that water, as well as hygienic problems that can subsequently result from the contact.

In one embodiment, a protective swatch may feature a waterproof, germ-impermeable, protecting swatch. On a top plane of the protective swatch, around its border, is a water-resistant, hypoallergenic, multi-use, skin-safe adhesive. A strategically-positioned, thin absorbent sanitary pad is featured on a top plane of the protective swatch. A wax paper may protect the adhesive and absorbent pad until use. The protective swatch may further feature a Comfort Gap that generally allows the protective swatch to bend and move without coming off or contacting the anus when the user walks or sits.

In other embodiments, a new and improved pathogen-impenetrable barrier that helps prevent vaginal infections by significantly covering and shielding a wearer's vagina from water and the contaminates within water, such as when the wearer is submerged, like while swimming and/or bathing. A pathogen-impenetrable shield that is comprised of an impermeable layer of pliable material, such as but not limited to rubber, plastic, silicon and/or any combination thereof, that is ergonomically shaped to comfortably wear between the legs and over the vagina. The pathogen-impenetrable barrier may feature a Comfort Gap in the form of a triangle in its bottom perimeter. The gap ensures the swatch of material doesn't make contact with the anus, and allows the swatch to bend without disengaging from wearer while the wearer walks and sits. Along the perimeter of one surface of the barrier is a water-resistant, hypoallergenic, multi-use, skin-safe adhesive which is protected by wax paper until use. The adhesive secures the barrier to the wearer and creates a seal between the water and vagina. The adhesive may be a pressure-sensitive, silicone-based adhesive. Situated lengthwise and centered upon the same surface side as the adhesive is an absorbent sanitary cloth for the purpose of absorbing any discharge from the wearer's vagina.

In additional embodiments, a base protecting swatch of the waterproof swatch is made of polyurethane, and in an hourglass-like shape with an approximate twelve-centimeter (12 cm) length. The width at the top-most end of the pad is approximately eight centimeters (8 cm), the width at the bottom-most endpoint is six centimeters (6 cm), and the narrowed center is of seven-centimeter (7 cm) width. There is an ergonomically-placed, triangular-shaped gap (known as the Comfort Gap) in the bottom perimeter of the swatch to prevent contact with the anus, and to allow movement without pulling when sitting and walking. Completely about the perimeter of the base protecting swatch on one (1) plane is a silicone gelatin adhesive. This adhesive area is approximately ½ wide. Affixed to the center of the same adhesive-bordered plane of the base pad is an absorbing pad. This absorbing pad is made of woven rayon material and features a top plane lining. This top plane lining is hydrophilic on its outer exterior plane, and hydrophobic on its inner interior plane. The absorbing pad is of the same hourglass-like design as the base pad, but in smaller dimensions of nine and one-half centimeters (9½ cm) in length, and with widths of three centimeters (3 cm) at its top-most end, two and one-half centimeters (2½ cm) at its bottom-most end.

In some other embodiments, the waterproof swatch may apply directly about the vulva. The waterproof swatch may encase the vulva with an impermeable, waterproof swatch. The waterproof swatch is intended for use by women in water environments. The waterproof swatch prevents water from a swimming environment from direct contact with the vulva and vagina. The waterproof swatch protects the vulva and vagina from exposure to chemicals commonly used in swimming pools, such as chlorine. By preventing contact with chlorine and other swimming pool chemicals, the waterproof swatch protects the normal bacterial flora of the vulva and vagina. By protecting normal bacterial flora, the waterproof swatch may eliminate a common cause of vaginal yeast infections. The waterproof swatch protects the vulva and vagina from infections that may be caused by contact with germs, bacteria, and parasites commonly found in natural bodies of water. The waterproof swatch protects the vulva and vagina from exposure to and contact with cryptosporidium, shigella, giardia, norovirus, tapeworm, hookworm, and other undesirable agents commonly found in natural bodies of water. The waterproof swatch protects the vulva and vagina from other infections that can be caused by contact with germs, bacteria, and parasites that can inhabit both swimming pool water and natural bodies of water, and both by natural exposure as well as from other swimmers. The waterproof swatch protects the vulva and vagina from exposure to and contact with Staphylococcus aureus, Escherichia coli, and other infectious bacteria. By protecting from such infections, the waterproof swatch allows users to remain in water recreation environments for longer time periods. By allowing users to remain in water recreation environments for longer periods of time, the waterproof swatch extends the duration of, and pleasure derived from, such water recreation environments. By protecting from such infections, the waterproof swatch eliminates the need for it users to bathe immediately after exiting water recreation environments. By eliminating the need to bathe immediately after exiting water recreation environments, the waterproof swatch allows users to engage in other activities in those environments. By protecting from such infections, the waterproof swatch may fill a gaping lapse in women's hygiene.

In further embodiments, while there are hygienic products and practices that treat the infections that can inflict women due to their presence in water environments, the waterproof swatch may actually prevent those infections. Instead of treating such common problems, the waterproof swatch may actually prevent these problems before they occur. By preventing these common problems before they occur, the waterproof swatch provides a unique and novel response. By preventing these common problems before they occur, the waterproof swatch may eliminate the need and expense of doctor visits and medications. By preventing these common problems before they occur, the waterproof swatch may initiate personal security in its users regarding water recreation environments. By initiating personal security regarding water environments, the waterproof swatch may encourage users to engage in such environments more frequently and for longer periods of time. By encouraging engagement in such environments more frequently and for longer periods, the waterproof swatch may improve the benefits, and aid personal goals, of physical exercise for its users. By encouraging engagement in such environments more frequently and for longer periods, the waterproof swatch may improve social interactions for its users.

Moreover, the waterproof swatch may also include an absorbent pad on its top-interior plane. The pad on the top-interior plane of the waterproof swatch may absorb light vaginal discharge. The waterproof swatch may protect others in the same water recreation environments of its users. The waterproof swatch may prevent bacteria, yeast discharge, and menstrual fluids from exiting the swimsuit worn by its user. By preventing bacteria, yeast discharge, and other elements from exiting the swimsuit of its use, the waterproof swatch may prevent acquisition of and infection from those elements by others sharing the same water environments. The waterproof swatch is of an ergonomic hourglass-like shape that provides comfortable accommodation to its point of intended placement. The waterproof swatch is thin and may be discreetly worn. The recommended adhesive of the waterproof swatch may maintain the pads secure and comfortable placement even during vigorous movements such as swimming. The recommended adhesive may allow removal and reapplication without damage to its adhering qualities, meaning the waterproof swatch can continue to provide its protective benefits even should it need to be temporarily removed outside of the water, such as to accommodate bathroom use. The waterproof swatch may provide all these same benefits in bathing environments.

In alternative embodiments, a waterproof swatch may be produced in various sizes and shapes, including but not limited to, sizes and shapes intended for use by children. The waterproof swatch can be made in various designs that would ensure stability in placement while accommodating full leg motions commonly used in water activities. The waterproof swatch can be made of various materials that are both flexible and impermeable by water, such as but not limited to polyurethane (PU), polyvinyl chloride (PVC), and silicone. The waterproof swatch may also be made of non-waterproof materials, such as but not limited to paper and nylon, but that still achieve waterproof status by spray application of a waterproofing agent, such as but not limited to silicone elastomer and expanded polytetrafluoroethylene (ePTFE). The waterproof swatch can be made of durable materials that are capable of multiple reuses. In such variations, the waterproof swatch may include an adhesive material in its final packaging, and which its users may apply for subsequent uses of the device. Also, in such variations of intended reusability, the waterproof swatch may include attachable absorbing pads for application upon its top plane. The waterproof swatch may use various hypoallergenic adhesive materials that are both waterproof and tolerable to skin contact, such as but not limited to silicone gelatin, methacrylates, epoxy diacrylates, and vinyl resins. The absorbing pad portion of the waterproof swatch may be made in various sizes and shapes. The absorbing pad portion of the waterproof swatch may be made of various applicable materials, including but not limited to rayon, cellulose wood pulp, and vicose. The top-exterior plane of absorbing pad portion of the waterproof swatch may be hydrophilic in order to draw any fluid substance within the pad, and/or may include a hydrophobic lining on the top-interior plane of the pad to prevent any escape of fluid substances it may have absorbed. The waterproof swatch can be made in variations that include scenting, and of various scent varieties, within its absorbing pad. The waterproof swatch can be made in variations that include no absorbing pad. The waterproof swatch can be made in variations in which its length and shape are altered in order to also cover the anus. The waterproof swatch may include a various number of units of the device in its final packaging. The waterproof swatch may include additional items relevant to its application, such as but not limited to moisturizing cream and depilatory cream, in its final packaging. The waterproof swatch can be made in variations of different sizes and shapes that are to be used to cover cuts and wounds. The waterproof swatch can be made in variations of different sizes and shapes that are used to cover other body parts such as but not limited to the breast. The waterproof swatch may be made in various colors and may bear, but not limited to, various patterns, designs and/or logos, which may or may not be of registered trademark and/or copyright status.

In use, a user may remove the wax paper lining of the waterproof swatch and then apply it over the vulva and vagina area, with the absorbing pad-hosting plane applied to the interior. She may then depress the adhesive-lined border to her skin and seal its placement. The user may then enjoy water activities in a swimming pool or other body of water, and without risk of that water contacting the vulva or vagina. Should the user need to temporarily remove the waterproof swatch, such as to allow bathroom use, she may simply remove it by hand, and then reapply the waterproof swatch immediately afterwards. Should there be light vaginal discharge or leakage during wear, the absorbing pad of the waterproof swatch may collect all of the discharge/leakage.

The present invention will now be described in detail with reference to embodiments thereof as illustrated in the accompanying drawings.

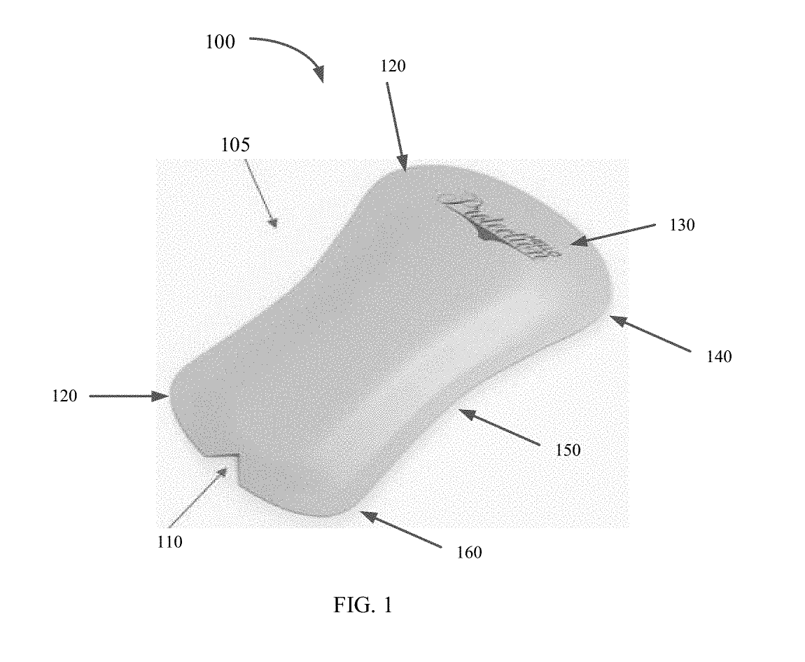

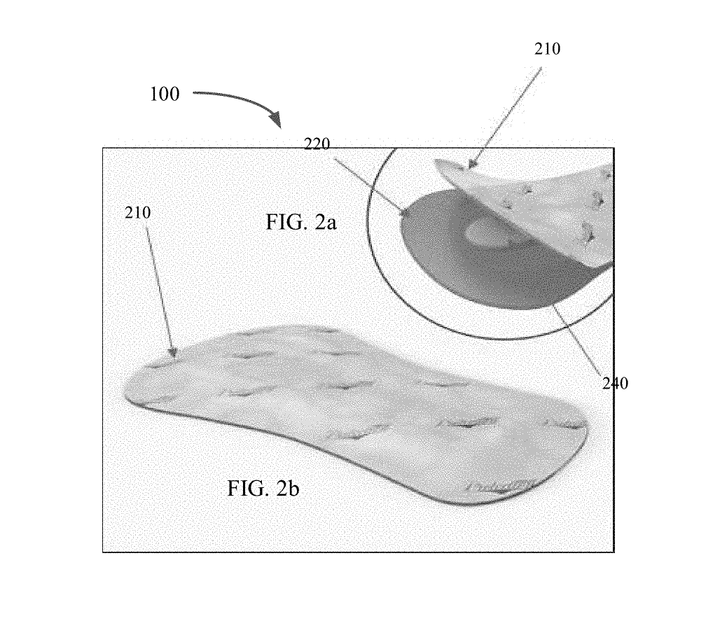

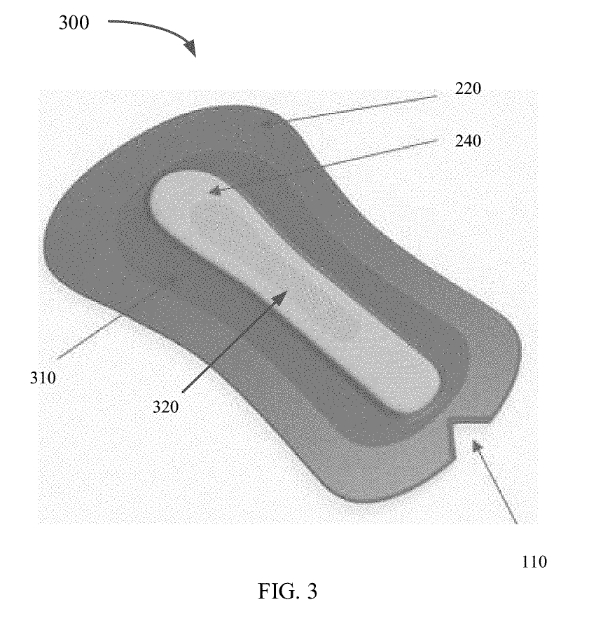

FIG. 1 is a front view illustration of an exemplary protective swatch 100, in accordance with an embodiment of the present invention. In the present embodiment, a front view of the protective swatch 100 shown. The protective swatch 100 may include a triangular-shaped gap or notch (comfort gap) 110 in a bottom perimeter 160 of the protective swatch 100 to significantly prevent contact with the anus, and to allow movement without pulling when sitting and walking. The protective swatch 100 may further include an ergonomic hourglass-like shape 105, with more or less twelve-centimeter (12 cm) length. The width at the top-most end 140 of the pad is approximately eight centimeters (8 cm), the width at the bottom-most endpoint 160 is about six centimeters (6 cm), and the narrowed center 150 is about seven-centimeter (7 cm) width. The corner portions 120 of the protective swatch 100 are configured to be round-shaped to avoid irritations caused by straight corners. The protective swatch 100 is water proof where the protective swatch 100 may be made of various materials that are both flexible and impermeable by water, such as but not limited to polyurethane (PU), polyvinyl chloride (PVC), rubber, plastic, and/or silicone. The protective swatch 100 may be made in various colors and may further include a display area 130 for bearing various patterns, designs, text, instruction, and/or logos. Alternatively, the protective swatch 100 may be made of different sizes and shapes to cover cuts and wounds and/or other body parts. The protective swatch 100 may also be made of non-waterproof materials, such as but not limited to paper and nylon, but that still achieve waterproof status by spray application of a waterproofing agent, such as but not limited to silicone elastomer and expanded polytetrafluoroethylene (ePTFE). The protective swatch 100 may also be made of durable materials that are capable of multiple reuses. In such variations, the waterproof swatch 100 may include an adhesive material in its final packaging, and which its users may apply for subsequent uses of the device. Also, in such variations of intended reusability, the waterproof swatch 100 may include attachable absorbing pads for application upon its top plane. A wax paper lining 210 (see FIG. 2) is applied to an adhesive 220 and absorbing pad 240 bearing plane of the base pad 300 (see FIG. 3). The wax paper lining 210 protects the adhesive 220 and absorbent pad 240 until use.

FIG. 2a and FIG. 2b is a back-view illustration of an exemplary protective swatch 100 with a protective covering, in accordance with an embodiment of the present invention. In the present embodiment shown, in FIG. 2a, a wax paper 210 is being peeled off the protective swatch 100, exposing an adhesive implement 220 and an absorbent pad 240. FIG. 2b shows the wax paper 210 covering the adhesive implement 220 and the absorbent pad 240 before the wax paper 210 is peeled off. The protective swatch 100 may use various hypoallergenic adhesive materials 220 that are both waterproof, tolerable to skin contact, hypoallergenic, multi-use, skin-safe, such as but not limited to silicone gelatin, methacrylates, epoxy diacrylates, and vinyl resins. The absorbing pad portion 240 of the protective swatch 100 may be made of various applicable materials, including but not limited to rayon, cellulose wood pulp, and vicose. The absorbing pad portion 240 of the protective swatch 100 may be made in various sizes and shapes. A top-exterior plane of absorbing pad portion 240 of the protective swatch 100 may be hydrophilic in order to draw any fluid substance within the absorbing pad portion 240, and/or may include a hydrophobic lining on a top-interior plane of the absorbing pad portion 240 to prevent any escape of fluid substances it may have absorbed. The absorbing pad portion 240 may include scenting, and of various scent varieties, within its absorbing pad. The protective swatch 100 may be made in variations in which its length and shape are altered in order to also cover the anus. The protective swatch 100 may include additional items relevant to its application, such as but not limited to moisturizing cream and depilatory cream, in its final packaging.

FIG. 3 is an illustration of an exemplary protective swatch base 300 ready for use, in accordance with an embodiment of the present invention. In the present embodiment shown, the wax paper 210 covering the adhesive implement 220 and the absorbent pad 240 was peeled off, exposing a base pad 300 of the protective swatch 100. Completely about the perimeter of the protective swatch base 300 on one (1) plane may include, but not limited to, a silicone gelatin, methacrylates, epoxy diacrylates, and vinyl resin adhesive 220. The adhesive area 220 is more or less half inch (½″) wide. Affixed to the center of the same adhesive-bordered plane of the base pad 300 is an absorbing pad portion 240. The absorbing pad portion 240 is made of woven rayon material and features a top plane lining 320 for the purpose of absorbing any discharge from the wearer's vagina. The top plane lining 320 is hydrophilic on its outer exterior plane, and hydrophobic on its inner interior plane. The absorbing pad portion 240 is of the same hourglass-like design as the base pad 300, but in smaller dimensions of more or less nine and one-half centimeters (9½ cm) in length, and with widths of about three centimeters (3 cm) at its top-most end, and about two and one-half centimeters (2½ cm) at its bottom-most end. The absorbent pad 240 may include scenting of various scent varieties. A non-adhesive portion 310 is disposed between the absorbing pad portion 240 and the adhesive implement 220. The non-adhesive portion 310 may be configured to be water and germ impermeable, however the non-adhesive portion 310 typically does not feature adhesive so as said adhesive does not actually make contact with the sensitive skin of the vulva. The adhesive implement 220 secures the protective swatch base 300 to a wearer and creates a seal between the water and vagina. The adhesive 220 may be a pressure-sensitive, silicone gelatin, methacrylates, epoxy diacrylates, and/or vinyl resin based adhesive.

In use, a user may remove the wax paper lining 210 of the waterproof swatch 100 and then apply the protective swatch base 300 over the vulva and vagina area, with the absorbing pad-hosting plane 240 applied to the interior. She may then depress the adhesive-lined border 220 to her skin and seal its placement. The user may then enjoy water activities in a swimming pool or other body of water, and without risk of that water contacting the vulva or vagina. Should the user need to temporarily remove the protective swatch base 300, such as to allow bathroom use, she may simply remove it by hand, and then reapply the protective swatch base 300 immediately afterwards. Should there be light vaginal discharge or leakage during wear, the absorbing pad 240 of the protective swatch base 300 may collect all of the discharge or leakage.

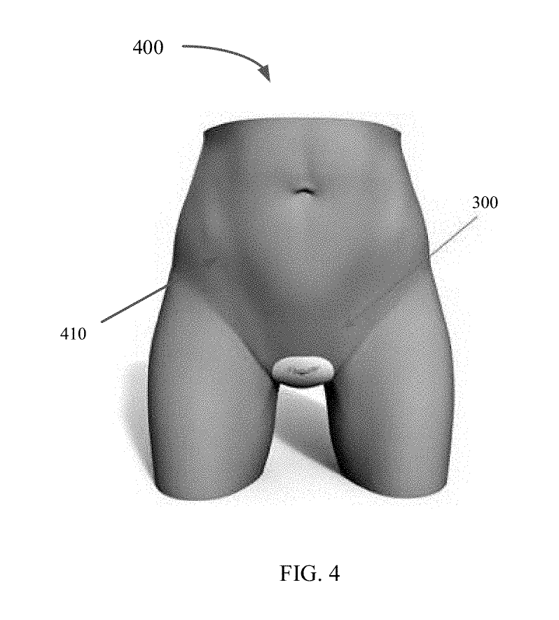

FIG. 4 is an illustration 400 of an exemplary protective swatch base 300 in use, in accordance with an embodiment of the present invention. In the present embodiment shown, the adhesive implement 220 secures the protective swatch base 300 to a wearer 410 and creates a seal between the water and vagina.

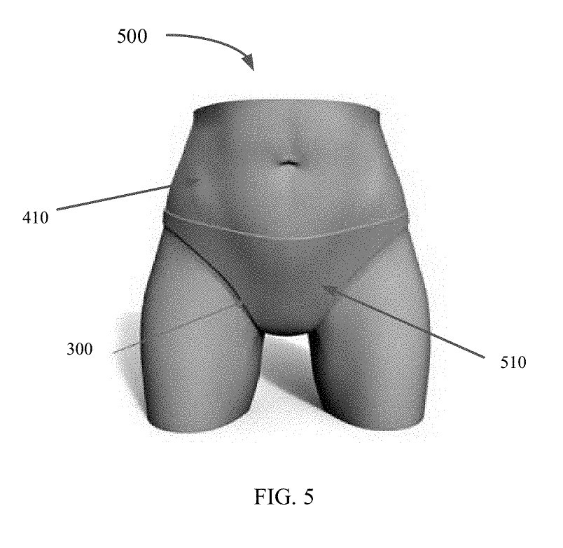

FIG. 5 is an illustration 500 of an exemplary protective swatch base 300 in use, in accordance with an embodiment of the present invention. In the present embodiment shown, the adhesive implement secures the protective swatch base 300 to a wearer 410 and creates a seal between the water and vagina. The protective swatch base 300 may remain inconspicuous when worn under a swimsuit 510 and may allow women to engage in water activities without being subjected to common hygienic risks.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(f)) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112(6) (post MA 112(f)) when such corresponding structures are not explicitly disclosed in the foregoing patent specification. Therefore, for any invention element(s)/structure(s) corresponding to functional claim limitation(s), in the below claims interpreted under 35 USC § 112(6) (post MA 112(f)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC § 112(6) (post MA 112(f)) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112(6) (post AIA 112(f)), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing protective swatches according to the present invention will be apparent to those skilled in the art. Various aspects of the invention have been described above by way of illustration, and the specific embodiments disclosed are not intended to limit the invention to the particular forms disclosed. The particular implementation of the protective swatch may vary depending upon the particular context or application. By way of example, and not limitation, the protective swatch described in the foregoing were principally directed to protecting the female private part implementations; however, similar techniques may instead be applied to cuts, wounds and/or other parts of the body, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

What is claimed is:

1. A protective implement comprising:
a swatch implement, wherein said swatch implement comprises a waterproof swatch base pad that is configured to be operable for preventing a private part of a wearer from exposure or contact to infections or disease caused by germs, bacteria, and parasites that inhabit swimming pool water or natural bodies of water;
wherein said waterproof swatch base pad comprises an ergonomic hourglass shape that is configured to encase the protected part of the wearer;
a triangular-shaped notch segment, said triangular-shaped notch segment is disposed in a proximate bottom perimeter of said waterproof swatch base pad, wherein said triangular-shaped notch segment is configured to be operable to allow movement without pulling when sitting and walking;
an adhesive implement disposed on a perimeter of said waterproof swatch base pad, in which said adhesive implement comprises at least one of, a silicone gelatin adhesive, methacrylates adhesive, epoxy diacrylates adhesive, and vinyl resin adhesive, wherein said adhesive implement is configured to secure said waterproof swatch base pad to the private part of the wearer;
an absorbent pad implement disposed on a proximate center of said waterproof swatch, wherein said absorbent pad implement is configured to absorb or prevent a discharge or menstrual fluid from the wearer from exiting said waterproof swatch base pad; and
a display area, said display area is configured to be operable for bearing at least one of, a pattern, a design, and a logo.

2. The protective implement of claim 1, further comprising a non-adhesive portion, wherein said non-adhesive portion is disposed between the absorbing pad portion and said adhesive implement.

3. The protective implement of claim 2, further comprising a wax paper lining, said wax paper lining is configured to protect said adhesive implement and absorbent pad implement until use, where said wax paper lining is peeled off during use.

4. The protective implement of claim 3, in which said ergonomic hourglass shaped swatch implement comprises round-shaped corner portions.

5. The protective implement of claim 4, in which said absorbent pad implement is made of woven rayon material and features a top plane lining.

6. The protective implement of claim 5, in which said top plane lining is hydrophilic on its outer exterior plane.

7. The protective implement of claim 6, in which said top plane lining is hydrophobic on its inner interior plane.

8. The protective implement of claim 1, in which said waterproof swatch is made of polyurethane.

9. The protective implement of claim 1, in which said absorbent pad implement comprises an hourglass shape.

10. The protective implement of claim 1, in which said adhesive implement is pressure-sensitive.

11. An implement consisting of:
means for preventing a private part of a wearer from exposure or contact from infections or disease caused by germs, bacteria, and parasites that inhabit swimming pool water or natural bodies of water;
means for allowing movement without pulling when sitting and walking;
means for securing said exposure preventing means to the private part of the wearer;
means for absorbing a discharge or menstrual fluid from exiting said exposure preventing means;
means for bearing at least one of, a pattern, a design, and a logo; and
means for protecting said securing means and said absorbing means until use.

12. An implement comprising:
a swatch implement, wherein said swatch implement comprises an ergonomic hourglass shape that is configured to encase a private part of a wearer;
wherein said swatch implement further comprises a waterproof swatch base pad that is configured to be operable for preventing the private part of the wearer from exposure or contact from infections or disease caused by germs, bacteria, or parasites that inhabit swimming pool water or natural bodies of water;
a triangular-shaped notch segment, said triangular-shaped notch segment is disposed in a proximate bottom perimeter of said waterproof swatch base pad, wherein said triangular-shaped notch segment is configured to be operable for allowing movement without pulling when sitting and walking;
an adhesive implement disposed around a perimeter of said waterproof swatch base pad, in which said adhesive implement comprises at least one of, a silicone gelatin adhesive, a methacrylates adhesive, an epoxy diacrylates adhesive, and a vinyl resin adhesive, wherein said adhesive implement is configured to secure said waterproof swatch base pad to the private part of the wearer, and in which said adhesive implement is pressure-sensitive;
an absorbent pad implement disposed on a proximate center of said waterproof swatch base pad, said absorbent pad implement is configured to absorb a discharge or menstrual fluid from the wearer from exiting said waterproof swatch base pad; and
a display area, said display area is configured to be operable for bearing at least one of, a pattern, a design, and a logo.

13. The protective implement of claim 12, further comprising a non-adhesive portion, wherein said non-adhesive portion is disposed between the absorbing pad portion and said adhesive implement.

14. The protective implement of claim 13, further comprising a wax paper lining, said wax paper lining is configured to protect said adhesive implement and absorbent pad implement until use, where said wax paper lining is peeled off during use.

15. The protective implement of claim 14, in which said ergonomic hourglass shaped swatch implement comprises round-shaped corner portions.

16. The protective implement of claim 15, in which said absorbent pad implement is made of woven rayon material and features a top plane lining.

17. The protective implement of claim 16, in which said top plane lining is hydrophilic on its outer exterior plane.

18. The protective implement of claim 17, in which said top plane lining is hydrophobic on its inner interior plane.

19. The protective implement of claim 12, in which said waterproof swatch is made of polyurethane.

20. The protective implement of claim 12, in which said absorbent pad implement comprises an hourglass shape.
